(19) United States
(12) Patent Application Publication
(10) Pub. No.: US 2016/0304477 A1
(43) Pub. Date: Oct. 20, 2016

(54) NOVEL METABOLITES OF VANOXERINE COMPOUNDS, PHARMACEUTICAL COMPOSITIONS CONTAINING THE SAME AND METHODS OF TERMINATING ACUTE EPISODES OF CARDIAC ARRHYTHMIA, RESTORING NORMAL SINUS RHYTHM, PREVENTING RECURRENCE OF CARDIAC ARRHYTHMIA AND MAINTAINING NORMAL SINUS RHYTHM IN MAMMALS

(71) Applicant: LAguna Pharmaceuticals, Inc., La Jolla, CA (US)

(72) Inventor: Arthur M. Brown, (US)

(21) Appl. No.: 14/781,284

(22) PCT Filed: Apr. 25, 2014
(86) PCT No.: PCT/US2014/035447

§ 371 Date: Sep. 29, 2015

Related U.S. Application Data
(60) Provisional application No. 61/816,214, filed on Apr. 26, 2013.
(60) Provisional application No. 61/905,039, filed on Nov. 15, 2013.

(51) Int. Cl. C07D 295/088

(52) U.S. Cl. CPC C07D 295/088

(57) ABSTRACT

Disclosed embodiments are related to pharmaceutical compositions comprising novel piperazine compounds and methods of administration of the same to a patient for termination acute episodes of cardiac arrhythmias, maintaining normal sinus rhythm, restoring normal sinus rhythm, and preventing recurrence of cardiac arrhythmia.

### FIELD OF THE INVENTION

Presently disclosed embodiments are related to novel piperazine compounds, pharmaceutical compositions comprising the same and methods for terminating acute episodes of cardiac arrhythmia, preventing the same, restoring normal sinus rhythm, preventing recurrence of cardiac arrhythmia, and maintaining normal sinus rhythm in mammals.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732). Vanoxerine has also been used for treating and preventing cardiac arrhythmia in mammals. (See U.S. Pat. No. 6,743,797 and U.S. Pat. No. 7,700,600).

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. In the US alone, AF currently afflicts more than 2.3 million people. By 2050, it is expected that there will be more than 12 million individuals afflicted with AF. While AF is rarely a lethal arrhythmia, it is responsible for substantial morbidity and can lead to complications such as the development of congestive heart failure or thromboembolism. Currently available Class I and Class III anti-arrhythmic drugs reduce the rate of recurrence of AF, but are of limited use because of a variety of potentially adverse effects, including ventricular proarrhythmia. Because current therapy is inadequate and fraught with side effects, there is a clear need to develop new therapeutic approaches.

Current first line pharmacological therapy options for AF include drugs for rate control. Despite results from several studies suggesting that rate control is equivalent to rhythm control, many clinicians believe that patients are likely to have better functional status when in sinus rhythm. Further, being in AF may introduce long-term mortality risk, where achievement of rhythm control may improve mortality.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, these agents have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Prior studies have been performed using single dose administration of flecainide or propafenone (Class I drugs) in terminating atrial fibrillation. Particular studies investigated the ability to provide patients with a known dose of one of the two drugs so as to self-medicate should cardiac arrhythmia occur. P. Alboni, et al., “Outpatient Treatment of Recent-Onset Atrial Fibrillation with the ‘Pill-in-the-Pocket’ Approach,” NEJM 351; 23 (2004); L. Zhou, et al., “‘A Pill in the Pocket’ Approach for Recent Onset Atrial Fibrillation in a Selected Patient Group,” Proceedings of UCLA Healthcare 15 (2011). However, the use of flecainide and propafenone has been criticized as including candidates having structural heart disease and thus providing patients likely to have risk factors for stroke who should have received antithrombotic therapy, instead of the flecainide or propafenone. NEJM 352:11 (Letters to the Editor) (Mar. 17, 2005). Similarly, the use of warfarin concomitantly with propafenone was criticized.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A Third Class of Anti-Arrhythmic Action: Effects on Atrial and Ventricular Intracellular Potentials and other Pharmacological Actions on Cardiac Muscle of MJ 1999 and AH 3747,” (Br. J. Pharmacol 39:675-689 (1970), and Singh B. N., Vaughan Williams E. M., “The Effect of Amiodarone, a New Anti-Anginal Drug, on Cardiac Muscle,” Br. J. Pharmacol 39:657-667 (1970)), but these are not selective Class III agents. Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)). Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (I_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{Kr} (rapidly activating and deactivating) and I_{Ks} (slowly activating and deactivating). (Sanguinetti and Jurkiewicz, “Two Components of Cardiac Delayed Rectifier K+Current. Differential Sensitivity to Block by Class III Anti-Arrhythmic Agents,” J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al., “A Molecular Basis for Cardiac Arrhythmia: hERG Mutations Cause Long QT Syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2, 4′-piperidin]-6yl], (+)-, monochloride (MK-499) predominantly, if not exclusively, block I_{Kr}. Although amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, as a nonspecific adrenergic blocker, acts as an inhibitor of the enzyme phospholipase, and causes pulmonary fibrosis (Nademanee, K., “The Amiodarone Odessey.” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness, by prolonging APD, prevents and/or terminates reentrant arrhythmias. Most selective Class III antiarrhythmic agents currently in development, such as d-sotalol and dofetilide predominantly, if not exclusively, block I_{Kr}, the rapidly activating component of I_{K} found both in atria and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M., “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (kJ potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supraventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced Effectiveness and Dangers of Reverse use Dependence,” Circulation, 81:686-690 (1990); Sadanaga et al., “Clinical Evaluation of the Use-Dependent QRS Prolongation and the Reverse Use-Dependent QT Prolongation of Class III Anti-Arrhythmic Agents and Their Value in Predicting Efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-Dependent Prolongation of Cardiac Action Potentials by a Methanesulfonanilide Class III Anti-Arrhythmic Agent: Specific Block of Rapidly Activating Delayed Rectifier K+ current by Dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

Vanoxerine has been indicated for treatment of cardiac arrhythmias. Indeed, certain studies have looked at the safety profile of vanoxerine and stated that no side-effects should be expected with a daily repetitive dose of 50 mg of vanoxerine. (U. Sogaard, et. al., “A Tolerance Study of Single and Multiple Dosing of the Selective Dopamine Uptake Inhibitor GBR 12909 in Healthy Subjects,” International Clinical Psychopharmacology, 5:237-251 (1990)). However, Sogaard, et. al. also found that upon administration of higher doses of vanoxerine, some effects were seen with regard to concentration difficulties, increase systolic blood pressure, asthenia, and a feeling of drug influence, among other effects. Sogaard, et. al. also recognized that there were unexpected fluctuations in serum concentrations with regard to these healthy patients. While they did not determine the reasoning, control of such fluctuations may be important to treatment of patients.

Further studies have looked at the ability of food to lower the first-pass metabolism of lipophilic basic drugs, such as vanoxerine. (S. H. Ingwersen, et. al., “Food Intake Increases the Relative Oral Bioavailability of Vanoxerine,” Br. J. Clin. Pharmac; 35:308-130 (1993)). However, no methods have been utilized or identified for treatment of cardiac arrhythmias in conjunction with the modulating effects of food intake.

In view of the problems associated with current anti-arrhythmic agents, there remains a need for new piperazine compounds, pharmaceutical compositions comprising the same, methods of manufacturing the same, and methods for treating patients with said compounds for the treatment of cardiac arrhythmias.

### SUMMARY

In accordance with these and other objects, a first embodiment of the disclosure comprises a piperazine compound having the following structure:

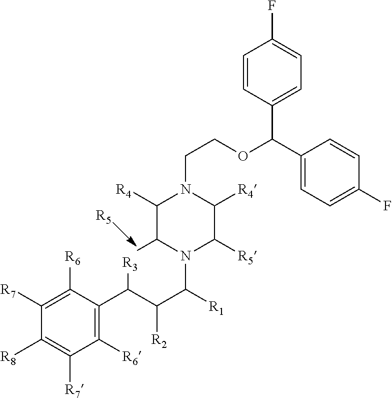

Wherein R1, R2, R3, R4/R4′, R5/R5′, R6/R6′, R7/R7′, or R8, are either a hydrogen atom or a hydroxide and further provided that not all of R1, R2, R3, R4/R4′, R5/R5′, R6/R6′, R7/R7′, or R8 are a hydrogen atom, and that only one of R1, R2, R3, R4/R4′, R5/R5′, R6/R6′, R7/R7′, or R8 is a hydroxide.

In further embodiments, one of R1, R4/R4′, R5/R5′, R6/R6′, R7/R7′, or R8 is a hydroxide and the remainder are a hydrogen atom. Still further embodiments, only one of R4/R4′ or R5/R5′ is a hydroxyl and the remainder hydrogen atoms. Still further embodiments consider wherein only one of R6/R6′ and R7/R7′ is a hydroxyl group and the remainder hydrogen atoms.

An additional embodiment of the present invention comprises pharmaceutical compositions containing one or more of the novel piperazine compounds shown above in admixture with a pharmaceutically acceptable carrier suitable for administration to a mammal.

An additional aspect of the present disclosure relates to a method of administration of the above structure comprising administering an effective amount of one or more of the novel piperazine compounds to a mammal for the treatment of cardiac arrhythmias.

An additional aspect of the present disclosure comprises methods for terminating acute episodes of cardiac arrhythmia, such as atrial fibrillation or ventricular fibrillation, in a mammal, such as a human, by administering to that mammal at least one of the novel piperazine compounds shown above in an amount effective to terminate an acute episode of cardiac arrhythmia.

An additional aspect of the present disclosure is directed to a method for restoring normal sinus rhythm in a mammal, such as a human, exhibiting cardiac arrhythmia by administering at least one of the novel piperazine compounds shown above in an amount effective to restore normal sinus rhythm.

An additional aspect of the present disclosure is directed to a method for maintaining normal sinus rhythm in a mammal, such as a human, by administering at least one of the novel piperazine compounds shown above in an amount effective to maintain normal sinus rhythm in a mammal that has experienced at least one episode of cardiac arrhythmia.

An additional aspect of the present disclosure is directed to a method for preventing a recurrence of an episode of cardiac arrhythmia in a mammal, such as a human, by administering to that mammal at least one of the novel piperazine compounds shown above in an amount effective to prevent a recurrence of cardiac arrhythmia.

A further embodiment is directed towards a method for treating cardiac arrhythmias comprising administering one or more of the novel piperazine compounds described herein wherein an effective amount of the compound is administered so as to reach a plasma concentration level of between about 20 and 200 ng/ml at a time between 0 and 4 hours post administration of one or more of the piperazine compounds.

Additional advantages, objects and feature of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

FIG. 1 depicts a chemical structure of the piperazine compounds described herein.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

As used herein, “administering” or “administer” refers to the actions of a medical professional or caregiver, or alternatively self-administration by the patient.

Cardiac arrhythmias include atrial, junctional, and ventricular arrhythmias, heart blocks, sudden arrhythmic death syndrome, and include bradycardias, tachycardias, re-entrant, and fibrillations. These conditions, including the following specific conditions: atrial flutter, atrial fibrillation, multifocal atrial tachycardia, premature atrial contractions, wandering atrial pacemaker, supraventricular tachycardia, AV nodal reentrant tachycardia, junctional rhythm, junctional tachycardia, premature junctional contraction, premature ventricular contractions, ventricular bigeminy, accelerated idioventricular rhythm, monomorphic ventricular tachycardia, polymorphic ventricular tachycardia, and ventricular fibrillation, and combinations thereof are all capable of severe morbidity and death if left untreated. Methods and compositions described herein are suitable for the treatment of these and other cardiac arrhythmias.

Administration of drug compounds leads to the metabolism and degradation of these compounds, often into a number of different compounds within the body. It is widely known that while a primary drug product may be administered, it may be one or more of the metabolic products that provides the efficacious drug. Similarly, one or more of the metabolic products may provide deleterious effects to the patient. Accordingly, it is advantageous to identify and isolate individual metabolites of advantageous drug products for administration to patients.

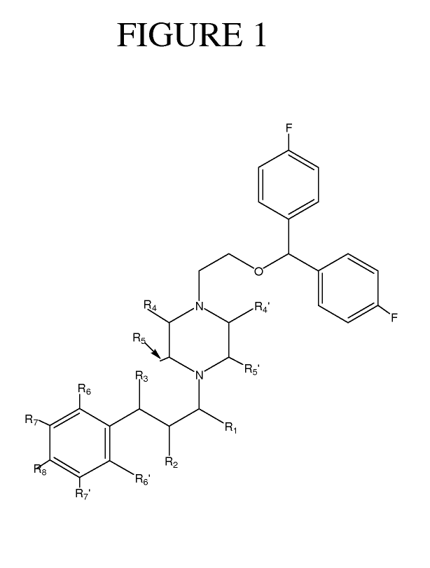

Accordingly, as depicted in FIG. 1 and below, the following structure identifies novel compounds for administration to a mammal for treatment of cardiac arrhythmias:

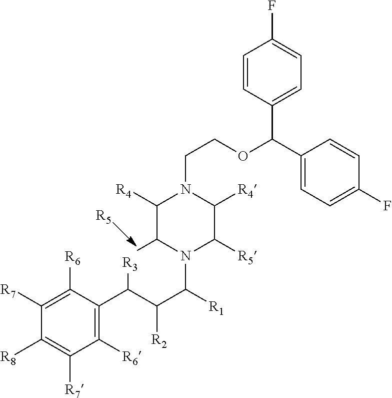

Wherein R1, R2, R3, R4/R4′, R5/R5′, R6/R6′, R7/R7′, or R8, are either a hydrogen atom or a hydroxide and further provided that not all of R1, R2, R3, R4/R4′, R5/R5′, R6/R6′, R7/R7′, or R8 are a hydrogen atom, and that only one of R1, R2, R3, R4/R4′, R5/R5′, R6/R6′, R7/R7′, or R8 is a hydroxide. Because of the nature of the molecule, R4 and R4′ for example, are chemically equivalent. Accordingly, only one of R4 or R4′ is a hydroxyl group and the other is a hydrogen atom. Similarly, R5 and R5′, R6 and R6′ and R7 and R7′ are equivalents of one another and thus one could be a hydroxyl and one a hydrogen atom.

Some piperazine compounds, including vanoxerine, are susceptible to metabolism by numerous mechanisms, including P450 and hepatic metabolism. Certain advantages exist by eliminating first pass metabolism by enzymatic catalysts and increasing the bioavailability with regard to a given dose of a piperazine compound. Accordingly, the bioavailability of a given dose of a piperazine compound can be increased by blocking and/or having a P450 antagonist to prevent the metabolism of vanoxerine before it has the ability to act on its intended target. This provides for either purely increased bioavailability per a given dose, or the opportunity to minimize a dose of a piperazine compound and still provide an efficacious dose of the drug compound.

In addition to metabolic processes, vanoxerine studies have shown that certain foods, whether high in fat or low in fat diet impact metabolism and bioavailability for some or all patients. (S. H. Ingwersen, et al., “Food Intake Increases the Relative Oral Bioavailability of Vanoxerine,” Br. J. Clin. Pharmac, 35:308-310 (1993)). However, even controlling for food, metabolic breakdown of vanoxerine still displays a high amount of variability with patient populations. Indeed, patients not only have different profiles with regard to metabolic variability of vanoxerine and certain metabolites, but also with regard to Cₘₐₓ and tₘₐₓ.

When providing treatment for patients, in some embodiments, the goal Cₘₐₓ plasma concentration may be about 5 ng/ml to about 1000 ng/ml of one or more of the novel piperazine compounds at one hour post administration. Indeed, for treatment of an acute episode of cardiac arrhythmia concentrations of about 5 ng/ml to about 400 ng/ml at one hour post administration are preferred. For preventative administration to prevent episodes of cardiac arrhythmia, concentrations of about 5 ng/ml to about 400 ng/ml at one hour post administration are preferred. For maintenance of normal sinus rhythm, concentrations of about 5 ng/ml to about 400 ng/ml at one hour post administration are preferred. For restoring normal sinus rhythm, concentrations of about 5 ng/ml to about 400 ng/ml at one hour post administration are preferred. For preventing recurrence of cardiac arrhythmia, concentrations of about 5 ng/ml to about 400 ng/ml at one hour post administration are preferred. Other preferred embodiments use a range of about 10 to about 250 ng/ml, about 20 to about 200 ng/ml, about 40 to about 200 ng/ml, about 40 to about 150 ng/ml, about 50 to about 150 ng/ml, and about 60 to about 125 ng/ml.

However, in other embodiments, it is important to maintain a plasma concentration of one of more of the novel piperazine compounds for a given time period, as measured by the mean concentration AUC. For example, it is advantageous to maintain a plasma level within the range of about 5 to about 400 ng/ml of one or more of the piperazine compounds for a period of about 1 to about 24 hours to restore normal sinus rhythm, or arrest an episode of cardiac arrhythmia. Doing so may require the administration of multiple doses over a given time period. Alternative embodiments utilize an elevated physiological level for more than a day. Indeed, it may be advantageous to provide for elevated physiological level for days, weeks, months, and/or years to maintain sinus rhythm and to prevent recurrence of cardiac arrhythmia. Accordingly a daily or multiple times a day dose may be appropriate in some circumstances for providing such elevated levels and to minimize fluctuation of levels due to pharmacokinetic metabolism.

In some embodiments, e.g. for the treatment of adult humans, a dosage of 1 mg to 1000 mg per unit dose is appropriate. Other embodiments may utilize a dosage of about 50 mg to 800 mg, or about 25 to about 100 mg, or about 100 mg to about 600 mg, or about 200 to about 400 mg.

Plasma level concentrations (and other physiological concentrations) are modified by the methods described herein. Preferred plasma level concentrations of one or more novel piperazine compounds, taken at a time point of 1 hour post administration are about 5 to about 400 ng/ml. In alternative embodiments, plasma level concentrations at 1 hour post administration are about 20 to about 200 ng/ml, or about 20 to about 150 ng/ml, or about 25 to about 125 ng/ml or about 40 to about 100 ng/ml, or about 60 to about 100 ng/ml. Alternatively, plasma level concentrations may be taken at a time point of about 90 minutes, 2 hours, 3 hours, 4 hours, 6 hours, 8 hours, 12 hours, and 24 hours post administration, or taken a multiple time points to provide additional data for maximizing the modifications in the methods described herein.

Additionally, modification of Cₘₐₓ and tₘₐₓ is appropriate to maintain consistent plasma level concentrations of one or more novel piperazine compounds. Cₘₐₓ taken at a time point of 1 hour post administration are about 5 to about 400 ng/ml. In alternative embodiments, plasma level concentrations at 1 hour post administration are about 10 to about 200 ng/ml, or about 20 to about 1200 ng/ml, or about 20 to 125 ng/ml, or about 25 to about 125 ng/ml or about 40 to 80 ng/ml. Conversely tₘₐₓ is appropriately reached at about 1 hour post administration. In other embodiments, tₘₐₓ is appropriately reached at about 30 minutes, or about 90 minutes, or about 120 minutes, or about 240 minutes post administration.

The novel piperazine compounds disclosed in the embodiments herein and the pharmaceutically acceptable salts thereof can be synthesized by conventional chemical methods using starting materials and reagents known and available to those skilled in the art. For example, with respect to pharmaceutically acceptable salts, generally, such salts are prepared either by ion exchange chromatography or by reacting the free base with stoichiometric amounts or with an excess of the desired salt-forming inorganic or organic acid in a suitable solvent or various combinations of solvents.

These compounds and pharmaceutically acceptable salts of the novel piperazine compound include, but are not limited to, salts of the novel piperazine compound formed from non-toxic inorganic or organic acids. Such pharmaceutically acceptable salts include, but are not limited to, the following: salts derived from inorganic acids, such as hydrochloric, hydrobromic, sulfuric, sulfamic, phosphoric, nitric and the like; salts derived from organic acids, such as acetic, propionic, succinic, glycolic, stearic, lactic, malic, tartaric, citric, ascorbic, pamoic, maleic, hydroxymaleic, phenylacetic, benzoic, salicylic, sulfanilic, 2-acetoxy-benzoic, fumaric, toluenesulfonic, methanesulfonic, ethane disulfonic, oxalic, isethionic, trifluoroacetic and the like; and salts derived from amino acids, such as glutamic acid or aspartic acid. See U.S. Pat. No. 6,187,802 and WO 91/01732.

When employed in the present methods, the piperazine compounds or a pharmaceutically acceptable salt, derivative or metabolite thereof, may be administered by any technique capable of introducing a pharmaceutically active agent to the desired site of action, including, but not limited to, buccal, sublingual, nasal, oral, topical, rectal and parenteral administration. Delivery of the compound may also be through the use of controlled release formulations in subcutaneous implants or transdermal patches. Administration may be with a bolus dose, or slow infusion, typically with the assistance of IV administration.

Suitable methods for treatment of cardiac arrhythmias include various dosing schedules. Dosing may include single daily doses, multiple daily doses, single bolus doses, slow infusion injectables lasting more than one day, extended release doses, IV or continuous dosing through implants or controlled release mechanisms, and combinations thereof. These dosing regimens in accordance with the method allow for the administration of piperazine compounds in an appropriate amount to provide an efficacious level of the compound in the blood stream or in other target tissues. Delivery of the compound may also be through the use of controlled release formulations in subcutaneous implants or transdermal patches.

For oral administration, a suitable composition containing the novel piperazine compound of the present invention, or a pharmaceutically acceptable salt thereof, may be prepared in the form of tablets, dragees, capsules, syrups, and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of the novel piperazine compound of the present invention, or a pharmaceutically acceptable salt thereof. Such tablets may, if desired, be provided with enteric coatings by known method, for example by the use of cellulose acetate phthalate. Suitable binding or granulating agents are e.g. gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing the novel piperazine compound of the present invention, or a pharmaceutically acceptable salt thereof, and at least one diluent, and optionally a part of the disintegrating agent, is granulated together with an aqueous, ethanolic or aqueous-ethanolic solution of the binding agents in an appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, a mixture of the novel piperazine compound of the present invention, or a pharmaceutically acceptable salt thereof, and the desired additives may be filled into a capsule, such as a hard or soft gelatin capsule. The contents of a capsule and/or caplet may also be formulated using known methods to give sustained release of the active compound.

Liquid oral dosage forms of the novel piperazine compound of the present invention, or a pharmaceutically acceptable salt thereof, may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing the novel piperazine compound of the present invention, or a pharmaceutically acceptable salt thereof, may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of the novel piperazine compound of the present invention, or a pharmaceutically acceptable salt thereof, may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspension may be prepared from sterile powders or granules having one or more carriers or diluents mentioned for use in the formulations for oral administration. Parenteral administration may be through intravenous, intradermal, intramuscular or subcutaneous injections.

A composition containing the novel piperazine compound of the present invention, or a pharmaceutically acceptable salt thereof, may also be administered nasally, for example by sprays, aerosols, nebulized solutions and/or powders. Metered dose systems known to those in the art may also be used.

Pharmaceutical compositions of the novel piperazine compound of the present invention, or a pharmaceutically acceptable salt thereof, may be administered to the buccal cavity (for example, sublingually) in known pharmaceutical forms for such administration, such as slow dissolving tablets, chewing gums, troches, lozenges, pastilles, gels, pastes, mouthwashes, rinses and/or powders.

Compositions containing the novel piperazine compound of the present invention, or a pharmaceutically acceptable salt thereof, for topical administration may comprise a matrix in which the pharmacologically active compound is dispersed such that it is held in contact with the skin in order to administer the compound transdermally. A suitable transdermal composition may be prepared by mixing the novel piperazine compound of the present invention, or a pharmaceutically acceptable salt thereof, with a topical vehicle, such as a mineral oil, petrolatum and/or a wax, for example paraffin wax or beeswax, together with a potential transdermal accelerant such as dimethyl sulphoxide or propylene glycol.

Alternatively, the novel piperazine compound of the present invention, or a pharmaceutically acceptable salt thereof, may be dispersed in a pharmaceutically acceptable cream or ointment base. The amount of the novel piperazine compound of the present invention, or a pharmaceutically acceptable salt thereof, contained in a topical formulation should be such that a therapeutically effective amount delivered during the period of time for which the topical formulation is intended to be on the skin.

The novel piperazine compound of the present invention, or a pharmaceutically acceptable salt thereof, may also be administered by continuous infusion either from an external source, for example by intravenous infusion or from a source of the compound placed within the body. Internal sources include implanted reservoirs containing the novel piperazine compounds of the present invention, or a pharmaceutically acceptable salt thereof, to be infused which is continuously released for example by osmosis and implants which may be (a) liquid such as a suspension or solution in a pharmaceutically acceptable oil of the compound to be infused for example in the form of a very sparingly water-soluble derivative such as a dodecanoate salt or (b) solid in the form of an implanted support, for example of a synthetic resin or waxy material, for the compound to be infused. The support may be a single body containing all the compound or a series of several bodies each containing part of the compound to be delivered.

The amount the novel piperazine compound of the present invention, or a pharmaceutically acceptable salt thereof, present in an internal source should be such that a therapeutically effective amount is delivered over a long period of time. In preferred embodiments, the piperazine compounds are administered at between 1 mg and 1000 mg, with additionally preferred doses at 25, 50, 100, 125, 150, 200, 300, 400, and 500 mg.

In addition, an injectable solution of the novel piperazine compound of the present invention, or a pharmaceutically acceptable salt thereof, can contain various additives such as preservatives, such as benzyl alcohol, methyl or propyl 4-hydroxybenzoate, benzalkonium chloride, phenylmercury borate and the like; as well as antioxidants, such as ascorbic acid, tocopherol, sodium pyrosulfate and optionally complex forming agents, such as an ethylenediamine tetraacetate salt for binding the metal traces, as well as buffers for adjusting the pH value and optionally a local anaesthetizing agent, e.g. lidocaine. The injectable solution containing the novel piperazine compound of the present invention, or a pharmaceutically acceptable salt thereof, is filtered before filling into the ampule and sterilized after filling.

With regard to oral formulations in particular, the novel piperazine compounds and methods of administration of the same may be carried out with the novel piperazine compound and optionally with certain excipients. The excipients are selected to ensure the delivery of a consistent amount of vanoxerine and to maintain plasma levels of the vanoxerine compound in a convenient unit dosage form and to optimize the dosing for the particular cardiac arrhythmia occurrence. All excipients must be inert, organoleptically acceptable, and compatible with vanoxerine. The excipients used in a solid oral formulation commonly include fillers or diluents, binders, disintegrants, lubricants, antiadherents, glidants, wetting and surface active agents, colors and pigments, flavoring agents, sweeteners, adsorbents, and taste-maskers.

Diluents may advantageously be added to a small amount of the active drug to increase the size of the tablet. A suitable diluent for use in the inventive compositions is lactose, which exists in two isomeric forms, alpha-lactose or beta-lactose, and can be either crystalline or amorphous. Various types of lactose include spray dried lactose monohydrate (such as Super-Tab™), alpha-lactose monohydrate (such as Fast Flo®), anhydrous alpha-lactose, anhydrous beta-lactose, and agglomerated lactose. Other diluents include sugars, such as compressible sugar NF, dextrose excipient NF, and dextrates NF. A preferred diluent is lactose monohydrate (such as Fast Flo®). Other preferred diluents include microcrystalline cellulose (such as Avicel® PH, and Ceolus™), and microfine cellulose (such as Elcema®).

Suitable diluents also include starch and starch derivatives. Starches include native starches obtained from wheat, corn, rice and potatoes. Other starches include pregelatinized starch NF, and sodium starch glycolate NF. Starches and starch derivatives can also function as disintegrants. Other diluents include inorganic salts, including, but not limited to, dibasic calcium phosphate USP (such as Di-Tab® and Emcompress®), tribasic calcium phosphate NF (such as Tri-Tab® and Tri-Cafos®), and calcium sulfate NF (such as Compactrol®). Polyols such as mannitol, sorbitol, and xylitol may also serve as diluents. Many diluents can also function both as disintegrants and as binders, and these additional properties should be taken into account when developing particular formulations.

Disintegrants may be included to break larger particles, such as tablets, granules, beads, nonpareils and/or dragees, into smaller particles comprising the active pharmaceutical ingredient and, optionally, other excipients which may facilitate dissolution of the active ingredient and/or enhance bioavailability of the active ingredient. Starch and starch derivatives, including cross-linked sodium salt of a carboxymethyl ether of starch (such as sodium starch glycolate NF, Explotab®, and Primogel®) are useful disintegrants. A preferred disintegrant is cross-linked sodium carboxymethyl cellulose (such as Croscarmellose Sodium NF, Ac-Di-Sol®). Other suitable disintegrants include, but are not limited to, cross-linked polyvinylpyrrolidone (such as Crospovidone NF) and microcrystalline cellulose (such as Avicel® PH).

Binders may also be used as an excipient, particularly during wet granulation processes, to agglomerate the active pharmaceutical ingredient and the other excipients. In all formulation, whether prepared by wet or dry granulation, a particular binder is generally selected to improve powder flow and/or to improve compactability. Suitable binders include, but are not limited to, cellulose derivatives, such as microcrystalline cellulose NF, methylcellulose USP, carboxymethylcellulose sodium USP, hydroxypropyl methylcellulose USP, hydroxyethyl cellulose NF, and hydroxypropyl cellulose NF. Other suitable binders include polyvidone, polyvinyl pyrrolidone, gelatin NF, natural gums (such as acacia, tragacanth, guar, and pectin), starch paste, pregelatinized starch NF, sucrose NF, corn syrup, polyethylene glycols, sodium alginate, ammonium calcium alginate, magnesium aluminum silicate and polyethylene glycols.

Lubricants may be used, particularly in tablet formulations, to prevent sticking of the ingredients and/or dosage form to the punch faces and to reduce friction during the compression stages. Suitable lubricants include, but are not limited to, vegetable oils (such as corn oil), mineral oils, polyethylene glycols (such as PEG-4000 and PEG-6000), salts of stearic acid (such as calcium stearate and sodium stearyl fumarate), mineral salts (such as talc), inorganic salts (such as sodium chloride), organic salts (such as sodium benzoate, sodium acetate, and sodium oleate) and polyvinyl alcohols. A preferred lubricant is magnesium stearate.

In preferred embodiments, the novel piperazine compound generally comprises from about 20-50% by weight of the pharmaceutical composition, more preferably from about 25-40% and most preferably from about 30-35%. Furthermore, suitable amounts of each excipient may be determined by one skilled in the art considering such factors as the particular mode of administration (e.g. oral, sublingual, buccal, etc.), amount of active ingredient (e.g. 50 mg, 60 mg, 80 mg, 100 mg, 150 mg, 200 mg, 400 mg, etc.), particular patient (e.g. adult human, human child, etc.) and dosing regimen (e.g. once a day, twice a day, etc.).

Solid dosage forms of a novel piperazine compound or combinations of such compounds can be prepared using any of the methods and techniques known and available to those skilled in the art.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

28 patients participated in a study of vanoxerine. 25 patients took a 300 mg dose of vanoxerine and 3 patients took a placebo. Each patient gave blood samples before administration of their dose, and then again at nine further time points, 30 minutes after admiration, 1, 2, 3, 4, 6, 8, 12, and 24 hours post administration.

Samples: A total of 270 human plasma samples were received (in duplicate). The samples were shipped frozen over dry ice. The samples were stored at −70° C. (nominal) until analysis.

Method of analysis outlines: Determination of vanoxerine and 17-hydroxyl vanoxerine concentrations in human plasma samples was performed against their respective calibration curve within concentration range of 1 to 250 ng/mL. The content determination of 16-hydroxyl vanoxerine and all other hydroxyl metabolites in human plasma samples was performed against 17-hydroxyl vanoxerine calibration curve within concentration range of 1 to 250 ng/mL.

Frozen samples were thawed at room temperature. Samples, calibrators, QC samples, sample blanks (blank plasma) and reagent blanks (water) were processed at room temperature. To 200 μL aliquots of each sample, calibrator, and QC sample are added 20 μL of 500 ng/mL of internal standard solution. A sample blank (blank plasma) and reagent blank (water) were also prepared, but without addition of internal standard solution (20 μL of diluent was added instead). To each sample, 200 μL of 1% ammonium hydroxide solution were added and vortex mixed. 3.0 mL of methyl tert-butyl ether were added and then vortex mix for 30 seconds. The sample is then centrifuged for 5 minutes at 4000 rpm and then transferred for about 30 minutes at −70° C. (nominal). The upper (organic) layer was transferred into an evaporation tubes and then evaporated under N₂ stream at ˜50° C. for about 20 min. Samples were then reconstituted in 1000 μL reconstitution solution (80:20:0.1 water: acetonitrile: formic acid). Samples were mixed, allow to stand for about 3 minutes and mixed again. 900 μL of the sample solution was transferred to a 1.5 mL microcentrifuge tube and centrifuged for 5 minutes at 14000 rpm at room temperature. 800 μL of the sample solution was transferred to a glass autosampler vial, and then transferred to the autosampler for analysis. Samples were separated using reversed-phase liquid chromatography with a C18 column.

Chromatographic conditions were as follows:

HPLC instrument: Waters Alliance e2795 HT with temperature controlled autosampler

Column: Waters XBridge C18 3.5μ 100×2.1 mm P.N. 186003022, Lot No. 0143302711 with an appropriate guard column

Column temperature: 45° C.

Autosampler temp: 5° C.

Flow: 0.3 mL/min

Mobile phase A: 10 mM ammonium formate buffer:MeOH:ACN 80:10:10

Mobile phase B: 10 mM ammonium formate buffer:MeOH:ACN 5:50:45

Purge solvent: Water:ACN:formic acid 50:50:0.1

Wash solvent: Water:ACN:formic acid 50:50:0.1

Injection volume: 20 μL

Run Time: 15 minutes

Gradient table:

| Time (min) | % A | % B | Curve |
|---|---|---|---|
| 0.00 | 100 | 0 | 1 |
| 0.20 | 100 | 0 | 6 |
| 2.00 | 50 | 50 | 6 |
| 6.00 | 50 | 50 | 6 |
| 6.10 | 0 | 100 | 6 |
| 10.90 | 0 | 100 | 6 |
| 11.00 | 100 | 0 | 6 |

Detection was based on electro-spray interface in positive mode (ESI+) LC-MS/MS technique, using Micromass Quattro Premier XE MS/MS detector with MassLynx and QuanLynx software version 4.1. MRM transitions for vanoxerine, 17-hydroxyl vanoxerine (M01) and for internal standard were m/z 451→203, 467→203 and 433→185 respectively. The MRM transition for all hydroxyl metabolites was m/z 467→203.

Calibration curve standards and QC samples were prepared by spiking vanoxerine, 17-hydroxyl vanoxerine and 16-hydroxyl vanoxerine into blank human plasma (with K₂EDTA as anticoagulant).

Calibration standards nominal concentrations of 1, 2, 10, 50, 100, 200 and 250 ng/mL and QC samples nominal concentrations of 3, 20, 125 and 187.5 were prepared for each analyte.

Results: The identification of the metabolites was as follows:

| | Retention time | Relative retention time |
|---|---|---|
| Compound | (min) | (RRT) |
| Vanoxerine | 9.21 | 1.00 |
| M03 | 7.38 | 0.81 |
| M04 | 7.88 | 0.86 |
| M01 (17-hydroxyl | 8.51 | 0.92 |
| vanoxerine) | | |
| M02 (16-hydroxyl | 8.74 | 0.95 |
| vanoxerine) | | |
| M05 | 8.96 | 0.97 |

TABLE 1
Concentrations ng/ml

| Time | | | | | | | Total |
|---|---|---|---|---|---|---|---|
| (h) | Vanoxerine | M03 | M04 | M01 | M02 | M05 | Metabolites |
| −15 | 1.00* | 1.00 | 1.00 | 1.00 | 1.00 | 1.00 | 1.00 |
| .5 | 25.26 | 1.02 | 10.79 | 1.93 | 1.00 | 1.30 | 12.44 |
| 1 | 70.09 | 2.46 | 49.74 | 7.51 | 1.02 | 1.88 | 60.41 |
| 2 | 104.98 | 7.08 | 82.62 | 19.65 | 1.02 | 2.59 | 111.20 |
| 3 | 81.43 | 7.21 | 75.63 | 18.68 | 1.01 | 2.14 | 102.83 |
| 4 | 54.30 | 7.54 | 63.85 | 16.42 | 1.01 | 1.45 | 88.35 |
| 6 | 32.85 | 6.59 | 48.14 | 11.48 | 1.00 | 1.22 | 66.35 |
| 8 | 24.37 | 4.92 | 38.38 | 8.98 | 1.00 | 1.21 | 52.45 |
| 12 | 15.89 | 3.98 | 26.84 | 6.30 | 1.00 | 1.05 | 37.05 |
| 24 | 8.29 | 2.32 | 13.46 | 3.66 | 1.00 | 1.01 | 19.07 |

*A quantity of (1) represents an amount that was below the lower limit of quantitation, which is <1.139 ng/ml vanoxerine, and <1.1141 ng/ml 17-hydroxyl vanoxerine.

TABLE 2
Standard Deviations

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 43.77 | 0.12 | 15.58 | 3.20 | 0.00 | 0.80 | 19.28 |
| 1 | 61.82 | 2.51 | 49.96 | 7.08 | 0.10 | 1.13 | 59.70 |
| 2 | 100.18 | 4.70 | 51.64 | 15.31 | 0.07 | 2.56 | 70.07 |
| 3 | 80.40 | 5.40 | 49.04 | 13.63 | 0.07 | 2.31 | 64.45 |
| 4 | 55.01 | 5.32 | 39.75 | 11.31 | 0.04 | 1.16 | 52.50 |
| 6 | 35.74 | 5.10 | 31.30 | 7.90 | 0.00 | 0.87 | 41.84 |
| 8 | 30.37 | 4.05 | 25.29 | 6.74 | 0.00 | 0.94 | 33.41 |
| 12 | 24.03 | 3.15 | 17.62 | 4.70 | 0.00 | 0.27 | 23.17 |
| 24 | 10.34 | 2.11 | 8.91 | 2.76 | 0.00 | 0.03 | 12.31 |

Table 2 shows the standard deviations from the above 25 patients receiving vanoxerine. The three patients receiving a placebo are not included in the data and all data points indicated levels of vanoxerine below the lower limit of quantitation.

Tables 1 and 2, above, show tests of 25 patients with a 300 mg dose of vanoxerine. Blood was drawn from each of the test patients before the administration of the vanoxerine, and then at 9 additional time points, one half hour after administration, then 1, 2, 3, 4, 6, 8, 12, and 24 hours subsequent to administration.

The 25 patients fall into two categories: 15 fell into a category of having the majority of time point levels that were below the average mean (as identified in Table 1) “low concentration group average,” and the remaining 10 patients had the majority of time points above the average mean “high concentration group average.”

TABLE 3
Low concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 16.99 | 1.00 | 12.17 | 1.52 | 1.00 | 1.37 | 13.39 |
| 1 | 40.07 | 2.78 | 56.35 | 6.76 | 1.03 | 1.73 | 66.46 |
| 2 | 42.50 | 6.48 | 74.06 | 14.09 | 1.00 | 1.30 | 94.80 |
| 3 | 31.40 | 5.36 | 59.58 | 11.38 | 1.00 | 1.14 | 76.25 |
| 4 | 24.40 | 5.91 | 51.98 | 10.34 | 1.00 | 1.05 | 68.14 |
| 6 | 16.69 | 4.96 | 38.61 | 7.08 | 1.00 | 1.00 | 50.52 |
| 8 | 11.82 | 3.29 | 29.92 | 5.30 | 1.00 | 1.00 | 38.45 |
| 12 | 6.31 | 2.58 | 20.60 | 3.67 | 1.00 | 1.00 | 26.71 |
| 24 | 5.01 | 1.79 | 12.09 | 2.66 | 1.00 | 1.00 | 16.08 |

TABLE 4
Low concentration standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 0.00 | 0.00 | 0.00 | 0.00 | 0.00 | 0.00 | 0.00 |
| 1 | 24.47 | 0.00 | 17.68 | 1.67 | 0.00 | 0.98 | 20.45 |
| 2 | 27.50 | 3.10 | 59.32 | 7.56 | 0.13 | 1.05 | 71.04 |
| 3 | 28.16 | 4.18 | 44.96 | 9.05 | 0.00 | 0.58 | 57.77 |
| 4 | 22.66 | 3.28 | 34.95 | 7.06 | 0.00 | 0.46 | 45.53 |
| 6 | 16.11 | 3.72 | 30.77 | 7.28 | 0.00 | 0.16 | 42.04 |
| 8 | 14.20 | 3.51 | 21.42 | 3.71 | 0.00 | 0.00 | 28.30 |
| 12 | 11.19 | 2.27 | 15.60 | 2.86 | 0.00 | 0.00 | 20.34 |
| 24 | 3.07 | 1.69 | 10.44 | 1.72 | 0.00 | 0.00 | 13.40 |

TABLE 5
High concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 37.67 | 1.06 | 8.71 | 2.55 | 1.00 | 1.19 | 11.01 |
| 1 | 115.12 | 1.98 | 39.82 | 8.64 | 1.00 | 2.10 | 51.33 |
| 2 | 198.71 | 7.96 | 95.46 | 28.00 | 1.05 | 4.51 | 135.79 |
| 3 | 156.49 | 9.98 | 99.70 | 29.64 | 1.03 | 3.64 | 142.69 |
| 4 | 96.14 | 9.83 | 80.45 | 24.93 | 1.02 | 2.01 | 116.64 |
| 6 | 57.08 | 9.03 | 62.44 | 18.08 | 1.00 | 1.55 | 90.10 |
| 8 | 43.18 | 7.37 | 51.08 | 14.50 | 1.00 | 1.52 | 73.46 |
| 12 | 29.30 | 5.93 | 35.57 | 9.98 | 1.00 | 1.13 | 51.52 |
| 24 | 3.07 | 1.69 | 10.44 | 1.72 | 0.00 | 0.00 | 13.40 |

TABLE 6
High concentration group standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 62.39 | 0.19 | 12.37 | 4.71 | 0.00 | 0.45 | 18.34 |
| 1 | 72.52 | 1.19 | 31.62 | 6.52 | 0.00 | 1.26 | 38.76 |
| 2 | 96.23 | 5.50 | 60.49 | 19.21 | 0.11 | 3.17 | 82.34 |
| 3 | 77.51 | 6.85 | 58.66 | 13.99 | 0.11 | 3.12 | 70.07 |
| 4 | 63.43 | 6.50 | 46.33 | 10.60 | 0.06 | 1.67 | 54.47 |
| 6 | 44.79 | 6.26 | 38.98 | 8.02 | 0.00 | 1.35 | 48.76 |
| 8 | 40.12 | 4.97 | 32.08 | 7.21 | 0.00 | 1.48 | 38.93 |
| 12 | 33.45 | 3.74 | 22.14 | 5.13 | 0.00 | 0.42 | 26.71 |
| 24 | 14.82 | 3.02 | 11.03 | 3.24 | 0.00 | 0.05 | 14.70 |

As can be seen, in Tables 3 and 5, the low concentration group barely has plasma levels rise above 40 ng/ml at any time point in reference to vanoxerine. Whereas, the high concentration group has levels that rise to nearly 200 ng/ml at a time of two (2) hours after administration. Furthermore, the variability with regard to each of the groups is also wider in the high group average. The standard deviations in Table 4 are lower than those in Table 6, (no T-test or 95% confidence was run), demonstrating that the variability was greater in the high concentration group than the low concentration group.

Modulation of a dose provides for greater accuracy with regard to target plasma concentrations for the treatment of cardiac arrhythmia. Utilization of certain methods allows for appropriate modulation of Cₘₐₓ and tₘₐₓ such that variability is minimized with patients. Furthermore, certain additional inhibitors may be advantageously utilized to improve first pass metabolism or bioavailability, such as P450 inhibitors. Furthermore, use of food modification, may, in certain instances provide additional consistency with regard to administration of piperazine compounds. Therefore, the methods provided for herein, provide for greater accuracy with regard to target physiological levels, thus increasing the safety profile, improving efficacy of treatment, and minimizing side effects that may be associated with treatment.

The metabolites M01, M02, M03, M04, and M05, are compounds generated by the body during metabolism of vanoxerine. What is evident with regard to the tables is the large standard deviations with regard to vanoxerine and certain of the metabolites. Furthermore, certain metabolites have a larger presence in the body than others, and accordingly may be primary or secondary compounds acting on the body in addition to vanoxerine. Therefore, administration of only one of, or a combination of these metabolites, and, alternatively in combination with vanoxerine, may lead to additional efficacy for the treatment of cardiac arrhythmias.

Certainly, in view of the relatively low bioavailability of vanoxerine, and the fact that there are significant accumulation of metabolites in the body, it is advantageous to directly administer certain metabolites of vanoxerine so as to increase their concentration in a patient, as opposed to indirectly administering them through administering vanoxerine and allowing the body's metabolism to generate the metabolites through the various metabolic pathways in the body.

Indeed, in preferred embodiments, concentrations in the plasma for one or more of the metabolites is between 1 and 1000 ng/ml at a time point of between 0 and 24 hours post administration of the piperazine compound. In particularly preferred embodiments, the concentration is between 10 and 400 ng/ml, or 20 and 200 ng/ml, or 40 and 150 ng/ml, or 60 and 120 ng/ml at 0-24 hours post administration. Furthermore, it is suitable to maintain these concentrations over the course of a day, more than a day, a week, or longer, wherein the concentration is measured as the mean area under the curve, which changes over time due to pharmacokinetic metabolism, based on the intake and the elimination of the drug via bodily mechanisms.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A piperazine compound having the following structure: 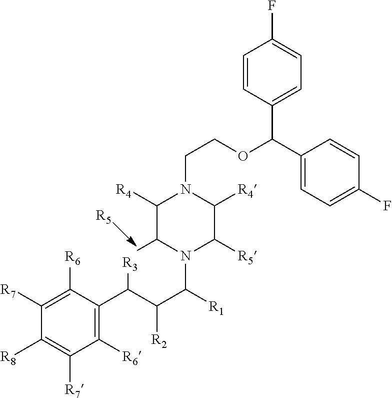
wherein R1, R2, R3, R4/R4′, R5/R5′, R6/R6′, R7/R7′, or R8, are either a hydrogen atom or a hydroxide and further provided that not all of R1, R2, R3, R4/R4′, R5/R5′, R6/R6′, R7/R7′, or R8 are a hydrogen atom, and that only one of R1, R2, R3, R4/R4′, R5/R5′, R6/R6′, R7/R7′, or R8 is a hydroxide.

2. The piperazine compound of claim 1 wherein one of R4 or R4′ is a hydroxide.

3. The piperazine compound of claim 1 wherein one of R5 or R5′ is a hydroxide.

4. The piperazine compound of claim 1 wherein one of R6 or 64′ is a hydroxide.

5. The piperazine compound of claim 1 wherein one of R7 or R7′ is a hydroxide.

6. The piperazine compound of claim 1 wherein R8 is a hydroxide.

7. A pharmaceutical composition comprising a piperazine compound having the following structure: 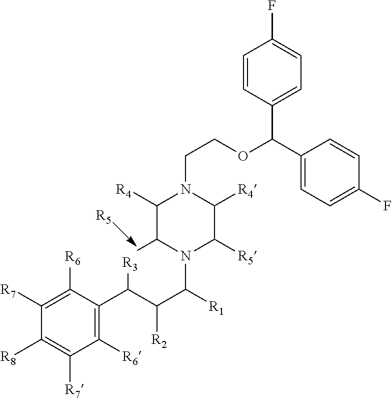
wherein R1, R2, R3, R4/R4′, R5/R5′, R6/R6′, R7/R7′, or R8, are either a hydrogen atom or a hydroxide and further provided that not all of R1, R2, R3, R4/R4′, R5/R5′, R6/R6′, R7/R7′, or R8 are a hydrogen atom, and that only one of R1, R2, R3, R4/R4′, R5/R5′, R6/R6′, R7/R7′, or R8 is a hydroxide, in admixture with a pharmaceutically acceptable carrier suitable for administration to a mammal.

8. The piperazine compound of claim 1 wherein one of R4 or R4′ is a hydroxide.

9. The piperazine compound of claim 1 wherein one of R5 or R5′ is a hydroxide.

10. The piperazine compound of claim 1 wherein one of R6 or 64′ is a hydroxide.

11. The piperazine compound of claim 1 wherein one of R7 or 74′ is a hydroxide.

12. The piperazine compound of claim 1 wherein R8 is a hydroxide.

13. A method of administration a pharmaceutical compound comprising the piperazine compound having the following structure: 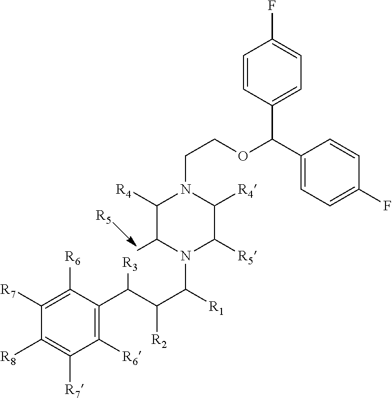
wherein R1, R2, R3, R4/R4′, R5/R5′, R6/R6′, R7/R7′, or R8, are either a hydrogen atom or a hydroxide and further provided that not all of R1, R2, R3, R4/R4′, R5/R5′, R6/R6′, R7/R7′, or R8 are a hydrogen atom, and that only one of R1, R2, R3, R4/R4′, R5/R5′, R6/R6′, R7/R7′, or R8 is a hydroxide, comprising the method of administering an effective amount of one or more of the novel piperazine compounds to a mammal for the treatment of cardiac arrhythmia.

14. The method of claim 13 wherein the cardiac arrhythmia is atrial fibrillation or ventricular fibrillation.

15. The method of claim 13 wherein the treatment of cardiac arrhythmia is restoring normal sinus rhythm in a mammal.

16. The method of claim 13 wherein the treatment of cardiac arrhythmia is an acute episode of cardiac arrhythmia.

17. The method of claim 13 wherein the effective amount of the one or more novel piperazine compounds results in a plasma concentration of said one or more piperazine compounds of between about 20 and about 200 ng/ml at one hour post administration.

18. The method of claim 13 wherein the effective amount of the one or more novel piperazine compounds results in a plasma concentration of said one or more piperazine compounds of between about 20 and about 150 ng/ml at one hour post administration.

19. The method of claim 13 wherein the effective amount of the one or more novel piperazine compounds results in a plasma concentration of said one or more piperazine compounds of between about 60 and about 125 ng/ml at one hour post administration.

20. The method of claim 13 wherein the effective amount of the one or more novel piperazine compounds results in a plasma concentration of said one or more piperazine compounds of between about 20 and about 200 ng/ml at between 0 and four hours post administration.
